(19) United States
(12) Patent Application Publication
(10) Pub. No.: US 2023/0404150 A1
(43) Pub. Date: Dec. 21, 2023

(54) Aerosol Generation Unit With Thermally Expandable Element for Controlling Liquid Supply

(71) Applicant: JT International S.A., Geneva (CH)

(72) Inventor: Alec Wright, Guildford (GB)

(73) Assignee: JT International S.A., Geneva (CH)

(21) Appl. No.: 18/034,835

(22) PCT Filed: Oct. 25, 2021
(86) PCT No.: PCT/EP2021/079500

§ 371 Date: May 1, 2023

(30) Foreign Application Priority Data
Nov. 5, 2020 (EP) 20206018.2

(51) Int. Cl. A24F 40/44; A24F 40/10; A24F 40/46; A24F 40/48; A24F 40/50

(52) U.S. Cl. CPC A24F 40/44; A24F 40/10; A24F 40/46; A24F 40/48; A24F 40/50

(57) ABSTRACT

An aerosol generation unit for use in an aerosol generation device or consumable includes a wicking element and a variable clamping element for controlling wicking of liquid by the wicking element. A The aerosol generation unit includes the wicking element in communication with a liquid reservoir, one or more clamping elements configured to thermally expand and contract to variably clamp at least a portion of the wicking element for controlling the amount of wicking, by the wicking element, of liquid contained in the reservoir, and a heating element or heat transfer element configured to apply heat to the wicking element and the one or more clamping elements. By variably clamping the wicking element, the capillary effect of the wicking element can be increased, reduced, or substantially suppressed. This allows the flow rate of liquid into the wicking element to be controlled based on the heat applied to the clamping element.

### FIELD OF INVENTION

The invention relates to an aerosol generation unit for use in an aerosol generation device or consumable. In particular, the invention relates to an aerosol generation unit with a wicking element and a variable clamping element for controlling wicking of liquid by the wicking element.

### TECHNICAL BACKGROUND

Aerosol generation devices and corresponding consumables that generate an aerosol from a liquid commonly utilize an aerosol generation unit that includes a capillary wicking element and a heating element arranged adjacent to the wicking element. The wicking element is in communication with a liquid reservoir and is commonly held in place either by a simple clamping means, or the heating element is shaped as a coil or spiral and the wicking element is held in place by being inserted through the hollow center of the heating element.

When in use, since the wicking element is in communication with the liquid reservoir, the wicking element has a capillary effect that draws and holds the drawn liquid, such that heating element can heat the held liquid to generate an aerosol. However, such a configuration has disadvantages. The wicking element draws liquid from the reservoir even when it is not used. As a result, drawn liquid may evaporate and become wasted. Drawn liquid can also leak out from the liquid reservoir through the wicking element and thus require the aerosol generation device or consumable to be cleaned or may damage the device or the consumable. Additionally, such a configuration is not capable of regulating or adjusting the amount of liquid drawn from the liquid reservoir and heated by the heating element based on the heating temperature.

Therefore, there is a need for an aerosol generation unit that includes a wicking element and a heating element that is capable of regulating a flow rate of liquid from a reservoir with which the aerosol generation unit is in communication, and that prevents liquid from being wasted or leaking from the liquid reservoir when the aerosol generation unit is not in use.

### SUMMARY OF THE INVENTION

Some or all of the above objectives are achieved by the invention as defined by the features of the independent claims. Preferred embodiments of the invention are defined by the features of the dependent claims.

A 1^{st} aspect of the invention is an aerosol generation unit for use in an aerosol generation device or consumable, the aerosol generation unit comprising a wicking element in communication with a liquid reservoir, one or more clamping elements configured to thermally expand and contract to variably clamp at least a portion of the wicking element for controlling the amount of wicking, by the wicking element, of liquid contained in the reservoir, and a heating element or heat transfer element configured to apply heat to the wicking element and the one or more clamping elements. By variably clamping the wicking element, the capillary effect of the wicking element can be increased, reduced, or substantially suppressed. This allows the flow rate of liquid into the wicking element to be controlled based on the heat applied to the clamping element.

According to a 2^{nd} nd aspect, in the preceding claim, the wicking element comprises or substantially consists of a fibrous and/or spongy material. A fibrous and/or spongy material is advantageous because of its strong capillary effect and because it can be variably clamped without being permanently altered or damaged.

According to a 3^{rd} rd aspect, in any one of the preceding aspects, the wicking element comprises or substantially consists of a cotton material, ceramic fiber, or glass fiber. These materials have the additional advantage of being heat resistant and prevent the wicking element from being burnt by heat applied by the heating element.

According to a 4^{th} aspect, in any one of the preceding aspects, the wicking element comprises or has an elongated shape. An elongated shape allows the wicking element to conform to the spatial requirements of aerosol generation devices or consumables commonly having an elongated shape.

According to a 5^{th} aspect, in any one of the preceding aspects, one or more ends of the wicking element are in communication with the liquid reservoir.

According to a 6^{th} aspect, in any one of the 4^{th} to 5^{th} aspects, the one or more clamping elements are arranged proximate to one or more ends of the wicking element that are in communication with the liquid reservoir. This increases the effectiveness of the regulation of the flow rate into the wicking element by the clamping elements.

According to a 7^{th} aspect, in any one of the preceding aspects, a clamping element forms a loop.

According to an 8^{th} aspect, in any one of the preceding aspects, a clamping element forms a ring.

A loop-shaped or ring-shaped clamping element allows the wicking element to further hold and/or fix a wicking element inserted through the hollow center of the clamping element. Additionally, a ring-shaped clamping element affords a uniform clamping of the wicking element.

According to a 9^{th} aspect, in any one of the preceding aspects, the one or more clamping elements comprise or substantially consist of a material with a coefficient of linear thermal expansion a between 1E-5 1/K and 1E-3 1/K, preferably between 1E-4 1/K and 1E-3 1 /K, most preferably between 5E-4 1/K and 1E-3 1/K. If the coefficient is too high, the sensitivity to temperature changes regarding the preferred expansions ranges within the spatial constraints of the aerosol generation unit is reduced. If the coefficient is too low, the expansion ranges become limited for sufficiently controlling the flow rate of liquid into the wicking element.

According to a 10^{th} aspect, in any one of the preceding aspects, the one or more clamping elements comprise or substantially consist of a material with a thermal conductance above

$$150 \frac{W}{m \cdot K} ,$$

preferably above

$$200 \frac{W}{m \cdot K} ,$$

more preferably above

$$250 \frac{W}{m \cdot K} ,$$

more preferably above

$$300 \frac{W}{m \cdot K} ,$$

most preferably above

$$350 \frac{W}{m \cdot K} .$$

This increases the thermal responsiveness of the expansion and contraction of the clamping elements.

According to an 11^{th} aspect, in any one of the preceding aspects, the one or more clamping elements comprise or substantially consist of polyamide, aluminum, copper, polyetherketoneketone (PEKK), or polyetheretherketone (PEEK).

According to a 12^{th} aspect, in the preceding claim, the one or more clamping elements comprise or substantially consist of polyamide 11.

The 11^{th} and 12^{th} aspects are advantageous due to their high tensile strength and temperature resistance.

According to a 13^{th} aspect, in any one of the preceding aspects, the one or more clamping elements are configured to compress or decompress at least a part of the wicking element based on their temperature. This allows the clamping element to be of a smaller size than the wicking element, since compressing or decompressing of only a part of the wicking element is sufficient for regulating the capillary effect of the wicking element.

According to a 14^{th} aspect, in any one of the preceding aspects, the one or more clamping elements are configured to at least locally compress the wicking element to substantially prevent wicking of liquid when they are not heated by the heating element or heat transfer element. This prevents liquid to flow into the wicking element when the aerosol generation device is not in use, and as a result prevents liquid from being wasted or leaking out from the wicking element and/or the liquid reservoir.

According to a 15^{th} aspect, in any one of the preceding aspects, the one or more clamping elements are configured to allow wicking of liquid when they are heated by the heating element or heat transfer element. This ensures that liquid only flows into the wicking element when needed when the aerosol generation is in use.

According to a 16^{th} aspect, in any one of the preceding aspects, the heating element or heat transfer element is arranged on at least a portion of the surface of the wicking element.

According to a 17^{th} aspect, in the preceding and the fourth aspect, the heating element or heat transfer element is arranged on at least a portion of a longitudinal surface of the wicking element.

The 16^{th} and 17^{th} aspects are advantageous because they improve the heating performance of the heating element when heating the wicking element, and further improve the application of heat to the one or more clamping elements for a more responsive control of wicking by the wicking element.

According to an 18^{th} aspect, in any one of the 16^{th} to 17^{th} aspects, the heating element or heat transfer element is wrapped or coiled around the at least a portion of the surface of the wicking element. This improves the heating performance of the heating element when heating the wicking element.

A 19^{th} aspect of the invention is an aerosol generation device for use with a consumable, the device comprising an aerosol generation unit according to any one the preceding claims.

According to a 20^{th} aspect, in the preceding aspect, the aerosol generation unit is arranged such that the wicking element is substantially perpendicular or parallel to the longitudinal extension direction of the aerosol generation device.

A 21^{st} aspect of the invention is a consumable for use with an aerosol generation device, the consumable comprising an aerosol generation unit according to any of the first to eighteenth aspects.

According to a 22^{nd} aspect of the invention, in any one of the preceding aspects, some or all of the one or more clamping elements are each an integrally formed element.

According to a 23^{rd} aspect of the invention, in the 14^{th} aspect, some or all of the one or more clamping elements are configured to at least locally compress the wicking element to securely hold the wicking element in place.

### BRIEF DESCRIPTION OF THE DRAWINGS

FIG. 1 shows a schematic illustration of an aerosol generation device and a consumable with an aerosol generation unit according to embodiments of the invention;

FIGS. 2A and 2B respectively show schematic cross-sectional illustrations of a portion of an aerosol generation unit according to embodiments of the invention;

FIGS. 3A and 3B show schematic perspective views of a portion of an aerosol generation unit according to embodiments of the invention;

FIGS. 4A, 4B and 4C show schematic illustrations of a portion of an aerosol generation unit with variable clamping elements according to embodiments of the invention;

FIGS. 5 shows a schematic illustration of an aerosol generation unit in communication with a liquid reservoir, according to embodiments of the invention.

### DETAILED DESCRIPTION OF PREFERRED EMBODIMENTS

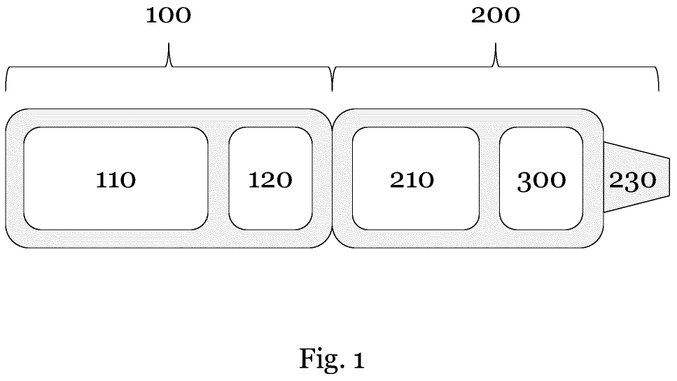

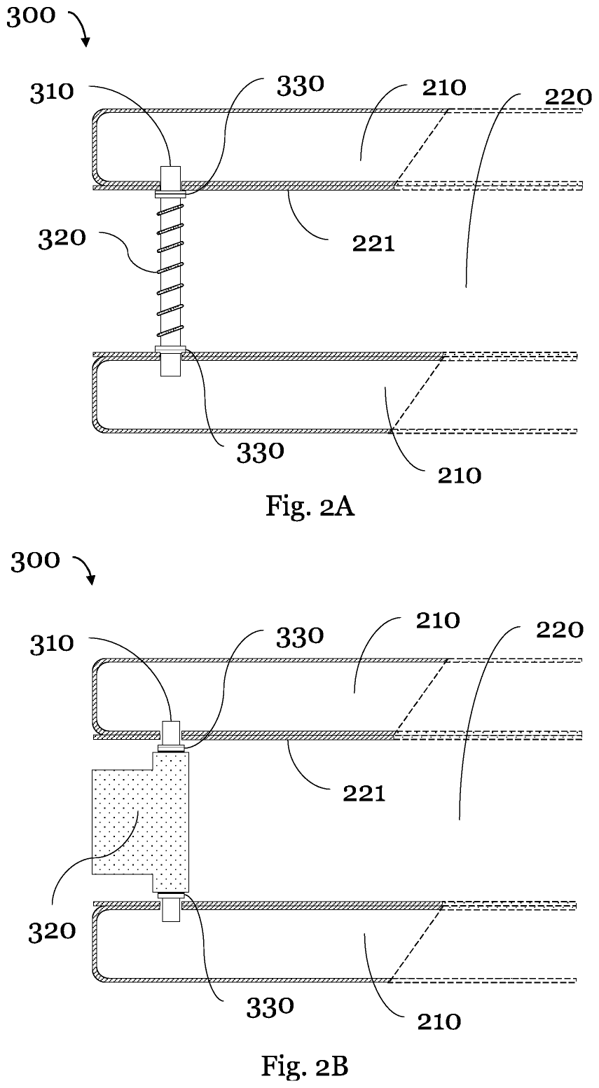

FIG. 1 illustrates an aerosol generation device 100 and a consumable 200 for use with the aerosol generation device 100. The aerosol generation device 100 comprises a power source 110 such as a rechargeable or exchangeable battery and may comprise circuitry 120. The circuity 120 may be configured for controlling a power supplied by the power source 110. The consumable 200 comprises a liquid reservoir 210 that contains a liquid for generating an aerosol. The consumable 200 may comprise an aerosol generation unit 30o that is configured to heat liquid from the liquid reservoir 210 to generate the aerosol. A mouthpiece 230 or similar outlet opening may be provided with the consumable for allowing the generated aerosol to be consumed by a user. It should be noted that while the aerosol generation device loo and its components and the consumable 200 and its components are illustrated as separate entities, each of the components may be configured for interacting with each other. For example, the circuitry 120 of the aerosol generation device wo may be configured for controlling the aerosol generation unit 30o comprised in the consumable 200. The power source 110 may be configured for supplying power to aerosol generation unit 300. Alternatively, instead of being comprised in the consumable 200, the aerosol generation unit 300 may be comprised in the aerosol generation device loo and configured to be in communication with the liquid reservoir 210 when the consumable is connected or attached to the aerosol generation device 100, and the mouthpiece 230 may alternatively be provided on the aerosol generation device 100. Further modifications with regard to which component or element may be comprised by the aerosol generation device loo or the consumable 200 are described below in the context of FIGS. 2 to 5.

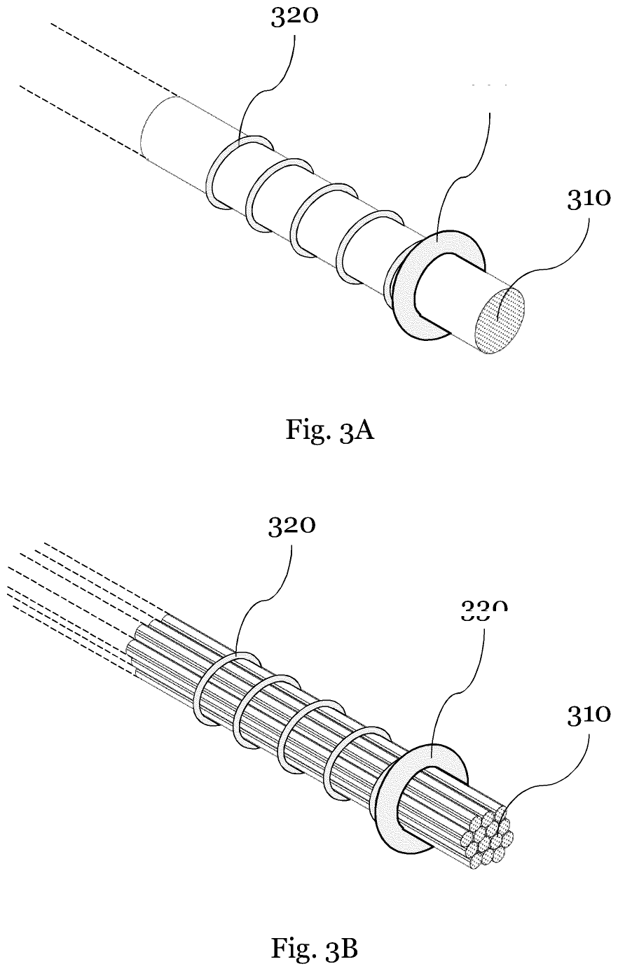

As shown in FIGS. 2A and 2B, an aerosol generation unit 30o comprises a capillary wicking element 310 that is arranged in communication with a liquid reservoir 210. The wicking element 310 may in general have one or more ends. In a preferred embodiment as exemplified in FIGS. 2A and 2B, the wicking element 310 has two ends. Preferably, the wicking element 310 is arranged transversely in the aerosol generation device loo or the consumable 200, i.e. the wicking element is arranged to be substantially perpendicular to the longitudinal extension direction of the aerosol generation device or the consumable. Additionally, the wicking element 310 may be a wicking element as described below for embodiments in the context of FIGS. 3A and 3B. The wicking element 310 is configured to be in communication with a liquid reservoir 210 with at least one of the one or more ends of the wicking element 310, preferably with each of the one or more ends of the wicking element 310 as shown in FIGS. 2A and 2B.

Preferably, the wicking element 310 is configured such that any liquid exiting the liquid reservoir 210 must flow into the wicking element 310. A clamping element 330 is provided at the wicking element 310, preferably at or proximate one or more ends of the wicking element 310 that are in communication with the liquid reservoir 210. A clamping element 330 is preferably provided at or proximate each of the one or more ends of the wicking element 310 that is in communication with the liquid reservoir 210.

As exemplified in FIG. 2A, a heating element 320 is arranged adjacent or at the wicking element 310 for heating the wicking element 310 and liquid drawn into the wicking element 310 as well as for heating each of the one or more clamping elements 330 provided at the wicking element 310. As exemplified in FIG. 2B, the heating element 320 may also be a passive heat transfer element that transfers heat from an active heating element to the wicking element 310. For example, as described for embodiments in the context of FIG. 1, the heating element 320 comprised by the consumable may instead be a passive heat transfer element, and the aerosol generation device 100 may comprise an active heating element configured for heating the heat transfer element when the consumable 200 is in use with the aerosol generation device. The liquid reservoir 210 may have a tubular or cylindrical shape with a hollow center. Additionally, the liquid reservoir 210 may be provided or filled with a liquid storage medium that comprises a fibrous or porous material. The aerosol generation unit 300 is arranged in or in communication with an airflow path 220 along which air flows from an air inlet to the mouthpiece 230 or air outlet. Preferably, a portion of the airflow path 220 extends through the hollow center in the axial direction of the hollow center. The portion of the airflow path 220 may be formed by a wall of the liquid reservoir 210 or may alternatively be formed by a central tubular element 221. Thus, aerosol generated by heating the liquid in the wicking element 310 may exit the aerosol generation device via the mouthpiece for consumption by a user.

When a clamping element 330 that is provided at or proximate one or more ends of the wicking element 310 is heated when the aerosol generation unit 300 is in use, the clamping element 330 is configured to expand based on its temperature 330 such that a clamping on the wicking element 310 is lessened, and wicking of liquid at the one or more ends is increased. When the heating of the wicking element 330 is subsequently reduced or stopped, the clamping element 330 is configured to contract based on its temperature such the clamping of the wicking element 310 is increased, and wicking of liquid at the one or more ends is decreased. Preferably, when the aerosol generation unit 300 is not in use and the clamping element 330 is not heated by the heating element 320, the clamping element 330 is configured to clamp the wicking element 310 such that substantially no wicking of liquid occurs. This prevents liquid from flowing into the wicking element 310 and from leaking out of the liquid reservoir when the aerosol generation unit 300 is not in use. Furthermore, under standard ambient conditions and when the aerosol generation unit 30o is not in use, the clamping element 330 may be configured to clamp the wicking element 310 such that the wicking element 310 is securely held by the clamping element 330. The clamping element 330 is preferably configured to clamp the wicking element 310, even in an expanded state of the clamping element 330 when it is heated to a predetermined maximum temperature, such that the wicking element 310 is securely held by the clamping element 330. In this way, the wicking element 310 can be securely held in the aerosol generation unit without requiring additional fastening means. The clamping of the clamping element 330 is thus based on its temperature and affords a temperature dependent control of the flow rate of liquid flowing into the wicking element 310.

FIGS. 3A and 3B each show a portion of an aerosol generation unit 300 that may be an aerosol generation unit as described above for embodiments in the context of FIGS. 1, 2A and 2B. In particular, FIGS. 3A and 3B illustrate a portion of the aerosol generation unit 30o near an end of a wicking element 310 comprised by the aerosol generation unit 300. The end of the wicking element 310 may be an end in communication with a liquid reservoir 210 as described for embodiments in the context of FIGS. 1, 2A and 2B. The aerosol generation unit further comprises a heating element 320 for heating the wicking element 310 and one or more clamping elements 330 for clamping at least a portion of the wicking element 310. The wicking element 310 may comprise or have an elongated shape or a rod like shape and may be straight or bent/curved. As shown in FIG. 3B, the wicking element 310 may comprise a plurality of elongated shapes bundled together to form the wicking element 310. It should be noted that while the wicking element 310 is shown to have a substantially circular cross-section, the wicking element 310 may have a rectangular, polygonal, or irregularly shaped cross-section. The wicking element 310 preferably comprises or consists of a spongy and/or fibrous material to ensure desired wicking characteristics of the wicking element. Such materials may comprise or consist of a cotton material, a glass fiber or ceramic fiber. These materials are reversibly deformable such that they adapt to variable clamping by the clamping element 330. These materials also provide a capillary effect for allowing wicking of liquid by the wicking element 310. It should be noted that the clamping element 330 may be provided at or proximate one or more ends of the wicking element 310, as described above in the context of FIGS. 1, 2A and 2B. In particular, if the wicking element 310 has two ends, the clamping element 330 may preferably be provided at or proximate each of the two ends.

The heating element 320 may be arranged proximate or in contact with the wicking element 310. The heating element 320 may be any appropriate type of heater. As shown, the heating element 320 may be coil or spiral shaped. Alternatively, the heating element 320 may be cylindrical, tubular or of a similar shape, or be in the form of a thin film heater. The heating element 320 is arranged proximate or on at least a portion of the outer surface of the wicking element 310. In case of an elongated or rod-like shaped wicking element 310, the heating element 320 is arranged proximate or on at least a portion of the longitudinal surface of the wicking element 310. Additionally, the heating element 320 may be arranged and dimensioned such that the heating element 320 is proximate or in contact with the clamping element 330. This allows the clamping element 330 to be responsive to heating by the heating element 320 without any substantial time delay and consequently leads to improved control of the flow rate of liquid.

The clamping element 330 is preferably arranged proximate or at an end of the wicking element 310 that is in communication with a liquid reservoir 210. While only a single clamping element 330 is shown, any appropriate number of clamping elements 330 may be provided at any appropriate position of the wicking element 310. The clamping element may be loop-shaped, ring-shaped, coil-shaped, cylindrical, tubular, U-shaped or have any shape suitable for clamping the wicking element 310. The clamping element 330 is configured to variably clamp the wicking element 310 based on the temperature of the clamping element 330. The clamping element 330 varies its clamping of the wicking element 310 based on its temperature by thermally expanding and contracting. By expanding and contracting, the wicking element 310 is compressed and decompressed at least locally at or proximate a position of the clamping element 330 at the wicking element 310. This allows the capillary effect of the wicking element 310 and thus the flow rate of liquid to be controlled by the clamping element 330.

The clamping element 330 may comprise or consist of a material with a suitable coefficient of linear thermal expansion α. The coefficient may be in the range of 1E-5 1/K and 1E-3 1/K, preferably between 1E-4 1/K and 1E-3 1/K, most preferably between 5E-4 1/K and 1E-3 1/K. If the coefficient is too high, the sensitivity to temperature changes regarding the preferred expansions ranges within the spatial constraints of the aerosol generation unit 300 is reduced. If the coefficient is too low, the expansion ranges become limited for sufficiently controlling the flow rate of liquid into the wicking element 310. Such materials may comprise or substantially consist of polyamide, preferably PA11, polyetherketoneketone (PEKK), polyetheretherketone (PEEK), or any suitable material with similar properties. Additionally, the clamping element 330 may comprise a material with a suitable thermal conductance. The thermal conductance may be above

$$150 \frac{W}{m \cdot K} ,$$

preferably above

$$200 \frac{W}{m \cdot K} ,$$

more preferably above

$$250 \frac{W}{m \cdot K} ,$$

more preferably above

$$300 \frac{W}{m \cdot K} ,$$

most preferably above

$$350 \frac{W}{m \cdot K} .$$

Such materials may comprise or substantially consist of aluminum, copper, or any suitable material with similar properties. In an embodiment, the clamping element 330 may comprise a first component comprising a material with a suitable coefficient of linear thermal expansion that is configured for causing the clamping element 330 to expand or contract based on its temperature. The clamping element 330 may further comprise a second component comprising a material with a suitable thermal conductance that is configured to increase conductance of heat from the heating element 320 or heat transfer element 320 to the first component for increasing the thermal response of the expansion or contraction of the clamping element 330. For example, the first component may comprise or substantially consist of PA11 and the second component may comprise or substantially consist of a copper material or aluminum material. The first and the second component may be attached or adjoined to each other. Preferably, the copper material or aluminum material may be provided as a copper or aluminum ring. The material with a suitable thermal conductance may be in thermal communication with the heating element 320 or heat transfer element 320. Alternatively, a material with a suitable coefficient of linear thermal expansion and a material with a suitable thermal conductance may be intertwined or interleaved to form a singular component. Alternatively, the clamping element 300 may substantially consist of only a material with a suitable coefficient of linear thermal expansion. Additionally, the clamping element 330 is preferably an integrally formed element.

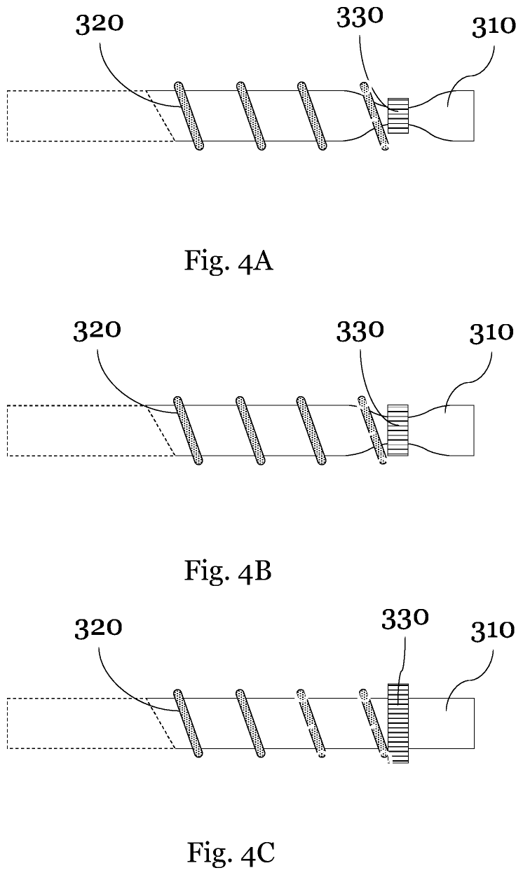

FIGS. 4A to 4C illustrate a portion of an aerosol generation unit 300 near an end of the wicking element 310 comprised by the aerosol generation unit 300. The aerosol generation unit 300 further comprises a heating element 320 and a clamping element 330 that is respectively shown to be in different clamping states in FIGS. 4A to 4C. The aerosol generation unit may be an aerosol generation unit 300 as described for embodiments in the context of FIGS. 1 to 3B. In particular, the wicking element 310 may have one or more ends that are in communication with a liquid reservoir 210, and a clamping element 330 may be provided at one or more of the ends that are in communication with the liquid reservoir 210. In a preferred embodiment, the wicking element 310 has two ends in communication with the liquid reservoir 210, and a clamping element 330 is provided at or proximate each of the two ends of the wicking element 330. The first state as exemplified in FIG. 4A may be a default or ambient state when the aerosol generation unit 30o is not in use. The wicking element 310 comprises an elongated shape, and the clamping element 330 is arranged such that its thermal expansion and contraction directions are substantially perpendicular to the (local) longitudinal direction of the wicking element 310. In this first exemplary state, the clamping element 330 is not heated by the heating element 320 and is not expanded. The clamping element 330 is sized such that, in this state, the wicking element 310 is compressed at least locally at or near the position of the clamping element 330 such that the capillary effect of the wicking element 310 is reduced. In this state, the capillary effect may be completely suppressed to prevent any wicking of liquid by the wicking element 310. This prevents any liquid from the liquid reservoir from being wasted or leaking out when the aerosol generation unit 300 is not in use. In a second exemplary state, as shown in FIG. 4B, the aerosol generation unit 300 is in use and the heating element 320 is heating the wicking element 310 and the clamping element 330. When heated, the clamping element 330 thermally expands and thus allows the wicking element to be more decompressed than in the first state. This removes the suppression of the capillary effect of the wicking element 310 and allows wicking of liquid by the wicking element 310. In a third exemplary state, as shown in FIG. 4C, the heating element 320 heats the wicking element 310 and the clamping element 330 to a higher temperature than in the second state. Due to the higher temperature, the clamping element 330 in the third state is more expanded than in the second state, thus allowing the wicking element 310 to be more decompressed than in the second state. Consequently, the capillary effect is increased and allows wicking of liquid by the wicking element 310 with a higher flow rate than in the second state. It should be noted that the states shown merely serve to illustrate the regulatory effect of the clamping element 330 on the wicking of liquid by the wicking element 310. It should be apparent to the skilled person that there may more than three states. In particular, there may be a continuous spectrum of temperature dependent states. This allows the flow rate to be finely and accurately controlled.

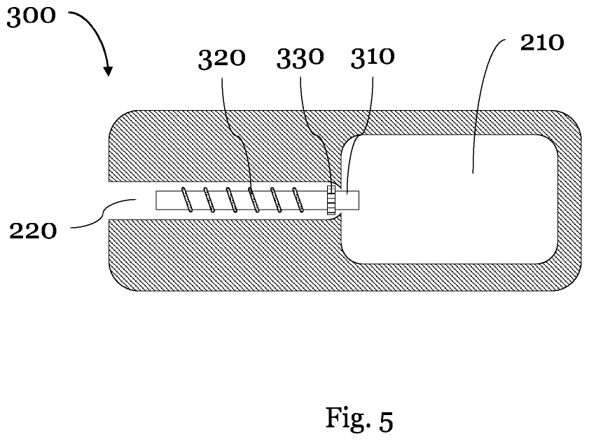

FIG. 5 shows modifications of embodiments described in the context of FIGS. 1 through 4C. For example, instead of a transversal arrangement of the wicking element 310 as shown in FIGS. 2A and 2B, the aerosol generation unit 300 may comprise a wicking element that is longitudinally arranged, i.e. the wicking element is arranged to be substantially parallel to the longitudinal extension direction of the aerosol generation device or the consumable. A longitudinal or transverse arrangement may be preferable based on the spatial constraints of the aerosol generation device loo or consumable 200 that comprises the aerosol generation unit 300. In particular, in case the longitudinally arranged wicking element 310 has two ends, only one end may be in communication with the liquid reservoir 210, and a clamping element 330 as described embodiments in the context of FIGS. 1 to 4C may be provided only at the one end that is in communication with the liquid reservoir 210. In any of the embodiments described above in the context of FIGS. 1 to 5, the wicking element 310 may also comprise or have a bent or curved shape instead of a straight shape.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the scope of this disclosure, as defined by the independent and dependent claims.

### LIST OF REFERENCE SIGNS USED

100: aerosol generation device
110: power source
120: circuitry
200: consumable
210: liquid reservoir
220: airflow path
221: central tubular element
230: mouthpiece
300: aerosol generation unit
310: wicking element
320: heating/heat transfer unit
330: clamping element

1. An aerosol generation unit for use in an aerosol generation device or consumable, the aerosol generation unit comprising:
a wicking element in communication with a liquid reservoir;
one or more clamping elements configured to thermally expand and contract to variably clamp at least a portion of the wicking element for controlling the amount of wicking, by the wicking element, of liquid contained in the reservoir; and
a heating element or heat transfer element configured to apply heat to the wicking element and the one or more clamping elements.

2. The aerosol generation unit according to the preceding claim 1, wherein the wicking element comprises a fibrous and/or spongy material.

3. The aerosol generation unit according to claim 1, wherein the wicking element comprises a cotton material, ceramic fiber, or glass fiber.

4. The aerosol generation unit according to claim 1, wherein the wicking element has an elongated shape, and/or wherein one or more ends of the wicking element are in communication with the liquid reservoir.

5. The aerosol generation unit according to claim 4, wherein the one or more clamping elements are arranged proximate to one or more ends of the wicking element that are in communication with the liquid reservoir.

6. The aerosol generation unit according to claim 1, wherein a clamping element forms a loop, and/or wherein a clamping element forms a ring.

7. The aerosol generation unit according to claim 1, wherein the one or more clamping elements comprise a material with a coefficient of linear thermal expansion α between 1E-5 1/K and 1E-3 1/K.

8. The aerosol generation unit according to claim 1, wherein the one or more clamping elements comprise a material with a thermal conductance above
$$150 \frac{W}{m \cdot K} .$$

9. The aerosol generation unit according to claim 1, wherein the one or more clamping elements comprise polyamide, aluminum, copper, Polyetherketoneketone (PEKK), or Polyetheretherketone (PEEK).

10. The aerosol generation unit according to claim 1, wherein the one or more clamping elements comprise or substantially consist of polyamide 11.

11. The aerosol generation unit according to claim 1, wherein the one or more clamping elements are configured to compress or decompress at least a part of the wicking element based on their temperature.

12. The aerosol generation unit according to claim 1, wherein the one or more clamping elements are configured to at least locally compress the wicking element to substantially prevent wicking of liquid when they are not heated by the heating element or heat transfer element, and/or wherein the one or more clamping elements are configured to allow wicking of liquid when they are heated by the heating element or heat transfer element.

13. The aerosol generation unit according to claim 1, wherein the heating element or heat transfer element is arranged on at least a portion of the surface of the wicking element.

14. An aerosol generation device for use with a consumable, the device comprising an aerosol generation unit according to claim 1.

16. A consumable for use with an aerosol generation device, the consumable comprising an aerosol generation unit according to claim 1.
